(19) United States
(12) Patent Application Publication
(10) Pub. No.: US 2019/0308037 A1
(43) Pub. Date: Oct. 10, 2019

(54) ULTRASOUND APPLICATOR WITH TEMPERATURE SENSOR

(71) Applicant: Barry Neil Silberg, Santa Rosa, CA (US)

(72) Inventor: Barry Neil Silberg, Santa Rosa, CA (US)

(21) Appl. No.: 15/793,000

(22) Filed: Oct. 25, 2017

(51) Int. Cl. A61N 7/02; A61B 5/00; A61B 5/055

(52) U.S. Cl. CPC A61N 7/02; A61B 17/2202; A61B 5/055; A61B 5/4836

(57) ABSTRACT

An ultrasound applicator designed to turn off an ultrasound system when an application area has reached too high of a temperature may include an ultrasound head; and a temperature sensor operatively attached to the ultrasound head and to the ultrasound delivery system. When the temperature sensor senses a temperature above a predetermined value, the ultrasound system may shuts off. The ultrasound applicator may additionally include an electronic module, wherein the temperature sensor is operatively connected to the electronic module which is operatively connected to the ultrasound deliver unit, and the electronic module includes a temperature controlled switch designed to turn off power to the ultrasound delivery unit when the temperature sensor senses the temperature above the predetermined value.

### BACKGROUND

The embodiments herein relate generally to medical devices, and more particularly, to an ultrasound applicator with a temperature sensor designed to shut down the ultrasound system when the application area reaches a particular temperature.

Currently, an ultrasound treatment cannot be performed on a sedated patient due to the possibility of skin burns and the inability of the sedated patient to provide feedback about burning skin.

Therefore, what is needed is an ultrasound applicator with a temperature sensor and control unit to sense the temperature of the application area and shut down the ultrasound system when unsafe temperatures are sensed, thus allowing an ultrasound treatment to be performed on an insensate patient or a patient under anesthesia.

### SUMMARY

Some embodiments of the present disclosure include an ultrasound applicator designed to turn off an ultrasound system when an application area has reached too high of a temperature may include an ultrasound head; and a temperature sensor operatively attached to the ultrasound head and to the ultrasound delivery system. When the temperature sensor senses a temperature above a predetermined value, the ultrasound system will shut off. The ultrasound applicator may additionally include an electronic module, wherein the temperature sensor is operatively connected to the electronic module which is operatively connected to the ultrasound deliver unit, and the electronic module includes a temperature controlled switch designed to turn off power to the ultrasound delivery unit when the temperature sensor senses the temperature above the predetermined value.

### BRIEF DESCRIPTION OF THE FIGURES

The detailed description of some embodiments of the invention is made below with reference to the accompanying figures, wherein like numerals represent corresponding parts of the figures.

FIG. 1 is a perspective/schematic view of one embodiment of the present disclosure.

FIG. 2 is a rear perspective view of one embodiment of the present disclosure.

FIG. 3 is a front bottom perspective of one embodiment of the present disclosure.

FIG. 4 is a cutaway view of one embodiment of the present disclosure, taken along line 4-4 in FIG. 1.

### DETAILED DESCRIPTION OF CERTAIN EMBODIMENTS

In the following detailed description of the invention, numerous details, examples, and embodiments of the invention are described. However, it will be clear and apparent to one skilled in the art that the invention is not limited to the embodiments set forth and that the invention can be adapted for any of several applications.

The device of the present disclosure may be used as an ultrasound applicator with a temperature sensor for use on all patients, including sedated and insensate patients, and may comprise the following elements. This list of possible constituent elements is intended to be exemplary only, and it is not intended that this list be used to limit the device of the present application to just these elements. Persons having ordinary skill in the art relevant to the present disclosure may understand there to be equivalent elements that may be substituted within the present disclosure without changing the essential function or operation of the device.

a. Ultrasound Applicator

b. Ultrasound Head

c. Temperature Sensor

The various elements of the device of the present disclosure may be related in the following exemplary fashion. It is not intended to limit the scope or nature of the relationships between the various elements and the following examples are presented as illustrative examples only.

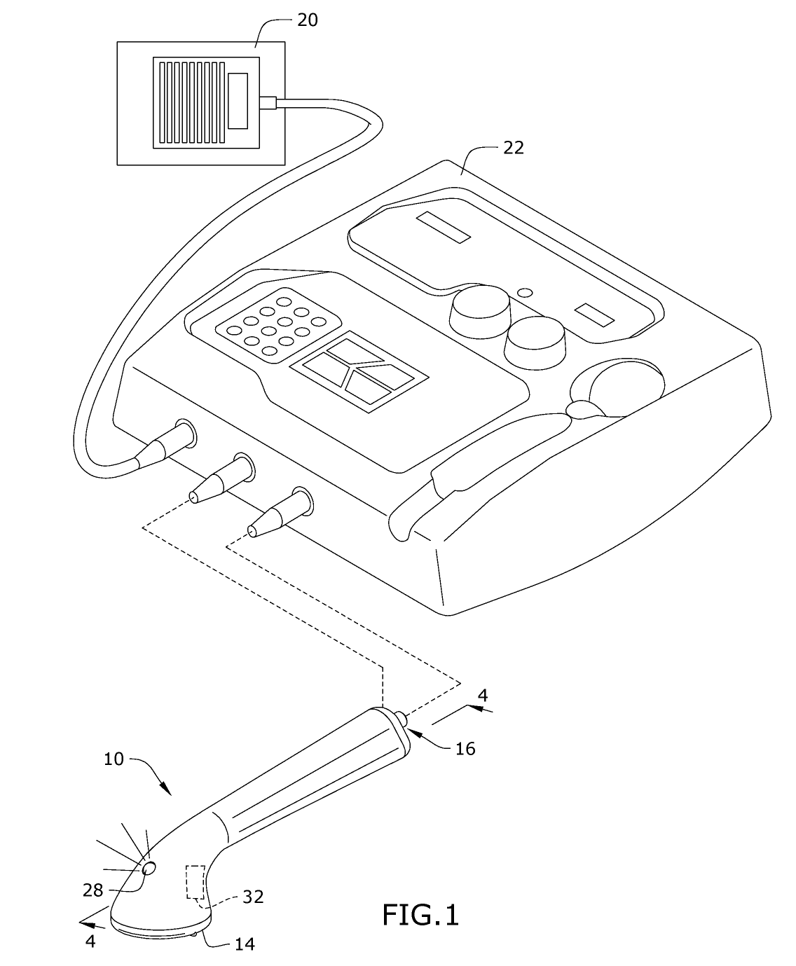

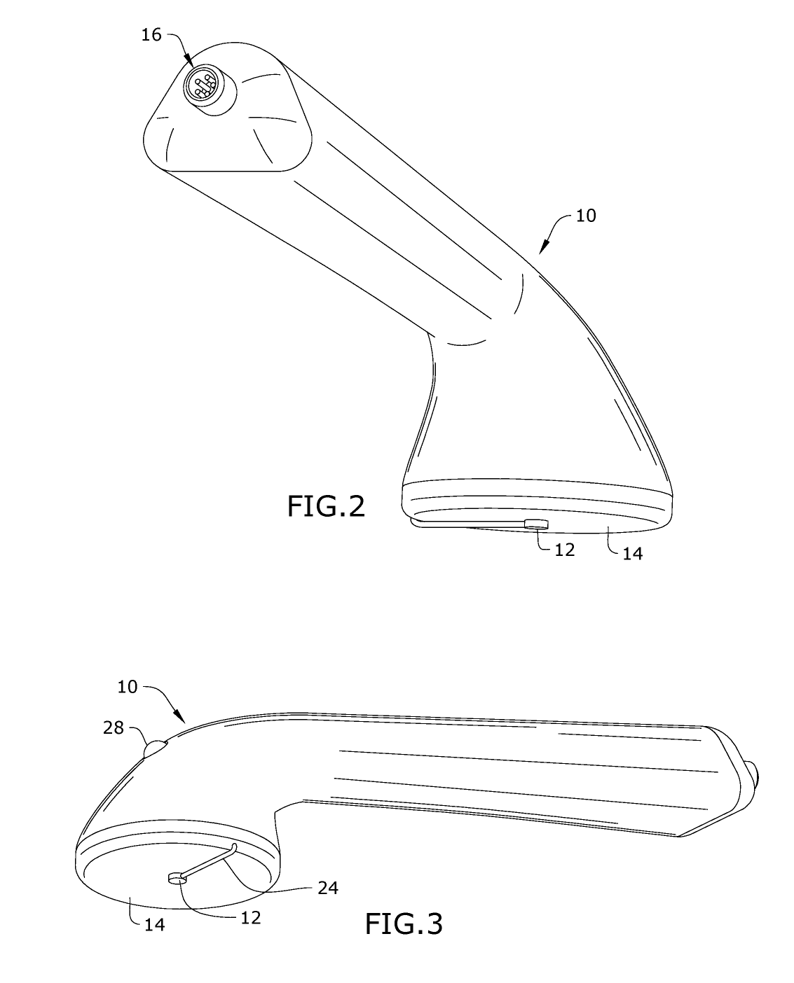

By way of example, and referring to FIGS. 1-4, some embodiments of the present disclosure include an ultrasound applicator 10 designed to turn off the ultrasound system when an application area has reached too high of a temperature, the ultrasound applicator 10 comprising an ultrasound head 14; and a temperature sensor 12 operatively attached to the ultrasound head 14, wherein the temperature sensor 12 is operatively connected to an ultrasound delivery unit 22 by, for example, a sensor connection 16. In embodiments, the temperature sensor 12 may be attached to an external surface of the ultrasound head 14. The temperature sensor 12 may also be attached to an electronic module 26 which may be operatively connected to the ultrasound delivery unit 22. The electronic module 26 may include a temperature controlled switch designed to turn off the power to the ultrasound delivery unit 22 upon the sensing of too high of a temperature on the ultrasound application area.

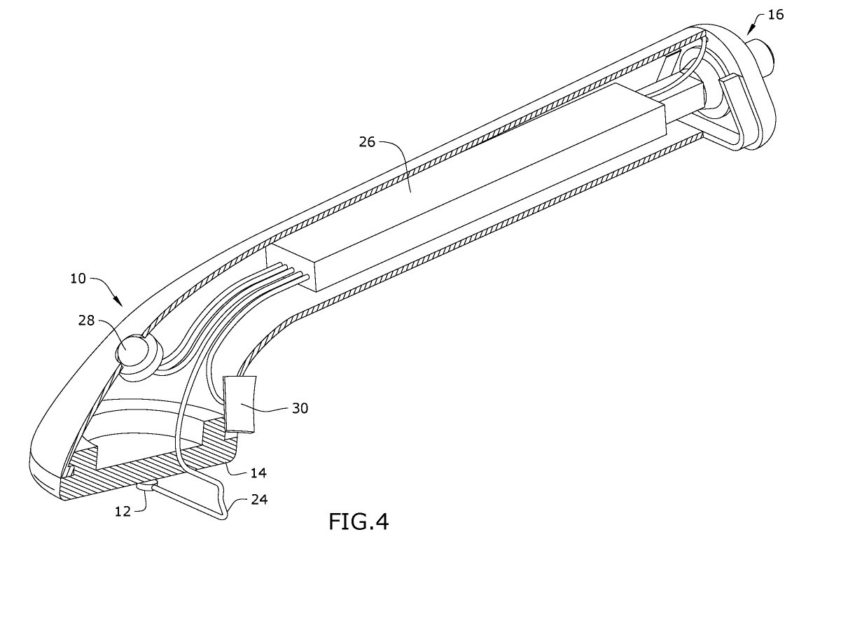

As shown, for example, in FIG. 4, the temperature sensor 12 may be connected to an electronic module 26 housed within the ultrasound applicator 10 by a sensor wire 24. The electronic module 26 may be attached to the ultrasound delivery unit 22 by the sensor connection 16. Additionally, a light emitting diode (LED) 28 may also be built into the ultrasound applicator 10 such that, when the ultrasound delivery unit 22 is powered on, the LED 28 may be illuminated. Lastly, a capacitive touch sensor 30 may also be built into the ultrasound applicator 10, wherein when a user touches the touch sensing location 32 on an exterior of the ultrasound applicator 10, the user may be able to control the ultrasound delivery unit 22. Alternatively, the ultrasound delivery unit 22 may be controlled by, for example, a foot switch 20. In either case, the sensing of a temperature by the temperature sensor 12 that is too high will override the operation of the ultrasound delivery unit 22 even if the foot switch 20 is pressed and/or the touch sensing location 32 is touched.

The temperature sensor 12 may be any suitable temperature sensing device, such as a thermocouple. When the temperature sensor 12 senses a temperature above, for example, about 48° C./118.4° F., which is the maximum temperature specified per the current Medical Device Standard for treatment time less than 10 minutes. As a result, the ultrasound applicator 10 may prevent overheating and possible burns to patients receiving external ultrasounds, particularly when they are under sedation or otherwise insensate and cannot give feedback to the person applying the ultrasound.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. An ultrasound applicator designed to turn off an ultrasound system when an application area has reached too high of a temperature, the ultrasound applicator comprising:
an ultrasound head; and
a temperature sensor operatively attached to the ultrasound head and to the ultrasound delivery system,
wherein when the temperature sensor senses a temperature above a predetermined value, the ultrasound system shuts off

2. The ultrasound applicator of claim 1, further comprising an electronic module, wherein:
the temperature sensor is operatively connected to the electronic module which is operatively connected to the ultrasound deliver unit; and
the electronic module includes a temperature controlled switch designed to turn off power to the ultrasound delivery unit when the temperature sensor senses the temperature above the predetermined value.

3. The ultrasound applicator of claim 1, wherein the temperature sensor is attached to an external surface of the ultrasound head.

4. The ultrasound applicator of claim 1, further comprising a light emitting diode (LED) built into the ultrasound applicator, wherein:
when the ultrasound applicator is transmitting ultrasound energy, the LED is illuminated.

5. The ultrasound applicator of claim 1, further comprising a capacitive touch sensor built into the ultrasound applicator.

6. The ultrasound applicator of claim 1, wherein the predetermined temperature at which the ultrasound system is shut off is about 48° C./118.4° F.
